(19) United States
(12) Patent Application Publication
(10) Pub. No.: US 2023/0154566 A1
(43) Pub. Date: May 18, 2023

(54) EPIGENETIC AGE PREDICTOR

(71) Applicant: H42, INC., Waarloos (BE)

(72) Inventors: Sandra Ann R. STEYAERT, San Diego, CA (US); Geert TROOSKENS, Meise (BE); Wim Maria R. VAN CRIEKINGE, Waarloos (BE); Adriaan VERHELLE, San Diego, CA (US); Johan Irma H. VANDERSMISSEN, Hoeselt (BE)

(73) Assignee: H42, INC., Waarloos (BE)

(21) Appl. No.: 17/831,430

(22) Filed: Jun. 2, 2022

Related U.S. Application Data
(63) Continuation-in-part of application No. 17/525,552, filed on Nov. 12, 2021.

(51) Int. Cl. G16B 30/00; G01N 33/92; A61B 5/145; A61B 5/00

(52) U.S. Cl. CPC G16B 30/00; G01N 33/92; A61B 5/14532; A61B 5/4872

(57) ABSTRACT

We propose an epigenetic age predictor and a method of training the same. The epigenetic age predictor is configured to receive a plurality of inputs corresponding to methylation values at CpG sites. The epigenetic age predictor is configured to receive a plurality of inputs corresponding to phenotypic values of an individual. The epigenetic age predictor predicts an epigenetic age of the individual based on the sequence of inputs.

### CROSS-REFERENCE TO RELATED APPLICATION

The present application is a continuation-in-part of and claims priority of U.S. patent application Ser. No. 17/525,552, filed Nov. 12, 2021, the content of which is hereby incorporated by reference in its entirety.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling and proteomics. Genomics arose as a data driven science, and operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks and many others.

Logistic regression is a binary classifier, that is, a supervised learning model that predicts a binary target variable. Specifically, logistic regression predicts the probability of the positive class by computing a weighted sum of the input features mapped to the [0, 1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation following by sequencing (ChlP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible kmers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATAI and TAL I. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATAI motif and TALI motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChlP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequence across distances as long as typical human introns (See Jaganathan, K. et al. Predicting splicing from primary sequence with deep learning. Cell 176, 535-548 (2019)).

Different types of neural network can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model at each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to find potential drivers of complex phenotypes. One example includes predicting the effect of noncoding single-nucleotide variants and short insertions or deletions (indels) indirectly from the difference between two variants in terms of transcription factor binding, chromatin accessibility or gene expression predictions. Another example includes predicting novel splice site creation from sequence or quantitative effects of genetic variants on splicing.

Human health and age can be measured in a variety of different ways. A human's chronological age, that is a time that an individual is alive, is one form of measure of a human's health and age. Another form of measuring a human's age is a subjective biological age that is used to account for a shortfall between a population average life expectancy and the perceived life expectancy of an individual of the same age. A human's environment or behavior can cause their body to biologically age at an accelerated rate. It has been difficult in the past to estimate an individual's biological age with accuracy.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 illustrates a diagram showing an example portion of DNA.

FIG. 2 illustrates a molecular structure diagram showing an example methylated cytosine molecule.

FIG. 3 illustrates a diagram showing an example portion of DNA.

FIG. 4A-4N illustrate diagrams showing genomic diagrams of chromosomes.

FIG. 5 illustrates a diagram showing an example array chip used to detect methylation.

FIG. 6 illustrates a diagram showing example beads that respond to methylated and unmethylated CpG sites.

FIG. 7 illustrates a flow diagram showing an example operation of training a model.

FIGS. 8-9 illustrate flow diagrams showing example operations of predicting an epigenetic age.

FIG. 10 illustrates a flow diagram showing an example operation of generating a phenotypic profile.

FIG. 11 illustrates a flow diagram showing an example operation of training a model.

FIGS. 12-14 illustrate flow diagrams showing example operations of predicting a modified epigenetic age.

FIG. 15 illustrates a block diagram showing an example computing system.

### DETAILED DESCRIPTION OF ILLUSTRATIVE EMBODIMENTS

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Many studies have shown that a human's environment or behavior can cause their body to biologically age at an accelerated rate. It has been difficult in the past to estimate an individual's biological age with accuracy. Genetics have been used with limited effectiveness as aging does not predictively correlate with alterations of the human DNA sequence. Epigenetics, however, have been shown to correlate with biological aging.

Epigenetics is the study of changes in gene expression that are not the result of changes in the DNA sequence itself. Some examples of processes that are in the field of epigenetics include acetylation, phosphorylation, ubiquitylation, and methylation. Methylation specifically, as we have found can correlate with a number of different diseases, conditions, the health of an individual as well as the biological age of the individual. Methylation can occur at several million different places in the human genome. Methylation can also differ from tissue to tissue and even cell to cell in the human body.

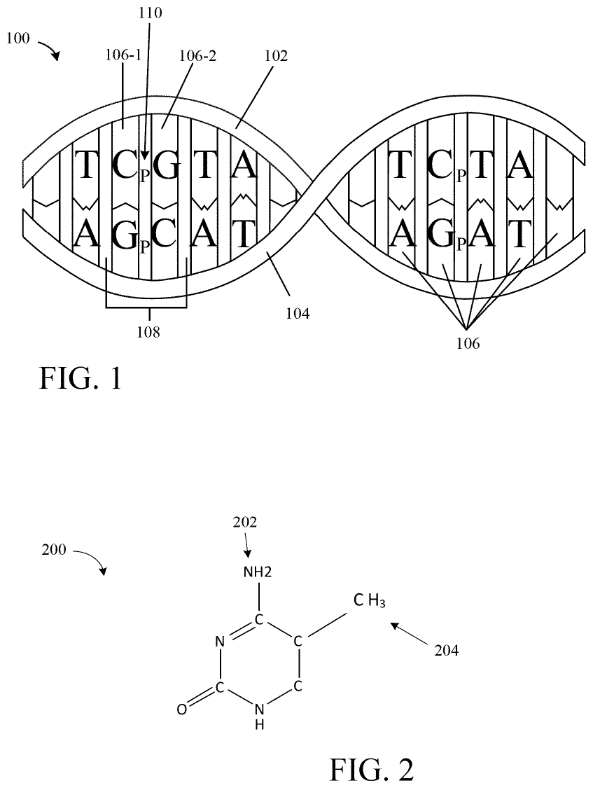

FIG. 1 is a diagram showing an example portion 100 of DNA. Portion 100 includes strand 102 and strand 104. Nucleotide base pairs 106 extend along the length of strands 102 and 104. An area where a cytosine nucleotide 106-1 is followed by a guanine nucleotide 106-2 is called a CpG site 108. CpG sites 108 are also defined in that only one phosphate group 110 separates the cytosine and quinine nucleotide base pairs 106.

FIG. 2 is a molecular structure diagram showing an example 5-Methylcytosine molecule 200. 5-Methylcytosine is an example of a methylated form of cytosine. Molecule 200 includes a cytosine portion 202 and a methyl group portion 204. As shown, the methyl group portion 204 is attached to the 5th atom in the 6-atom ring, counting counterclockwise from the NH-bonded nitrogen at the six o'clock position of the cytosine portion 202.

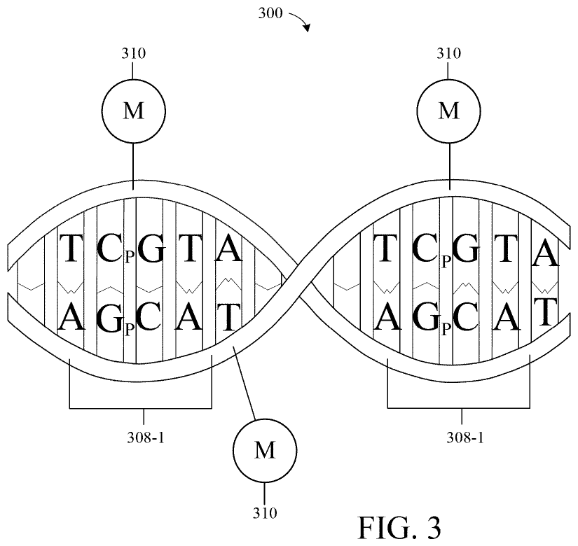

FIG. 3 is a diagram showing an example portion 300 of DNA. Portion 300 includes two CpG sites 308-1 and 308-2 (collectively referred to as CpG sites 308). The first CpG site 308-1 has two methyl groups 310 attached to the two cytosine molecules. This configuration of CpG site 308-1 is called being fully methylated. The second CpG site 308-2 has a single methyl group 310 attached to one of the two cytosine molecules. This configuration of CpG site 308-2 is called being hemi-methylated. When there are no methyl groups attached to either cytosine, then the CpG site is considered to be unmethylated.

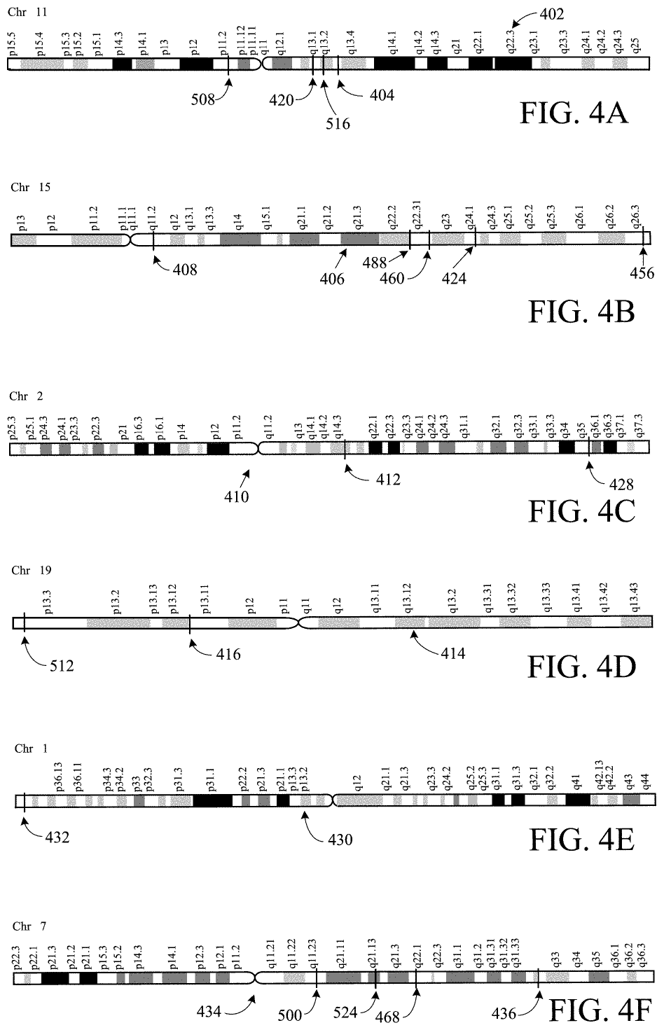

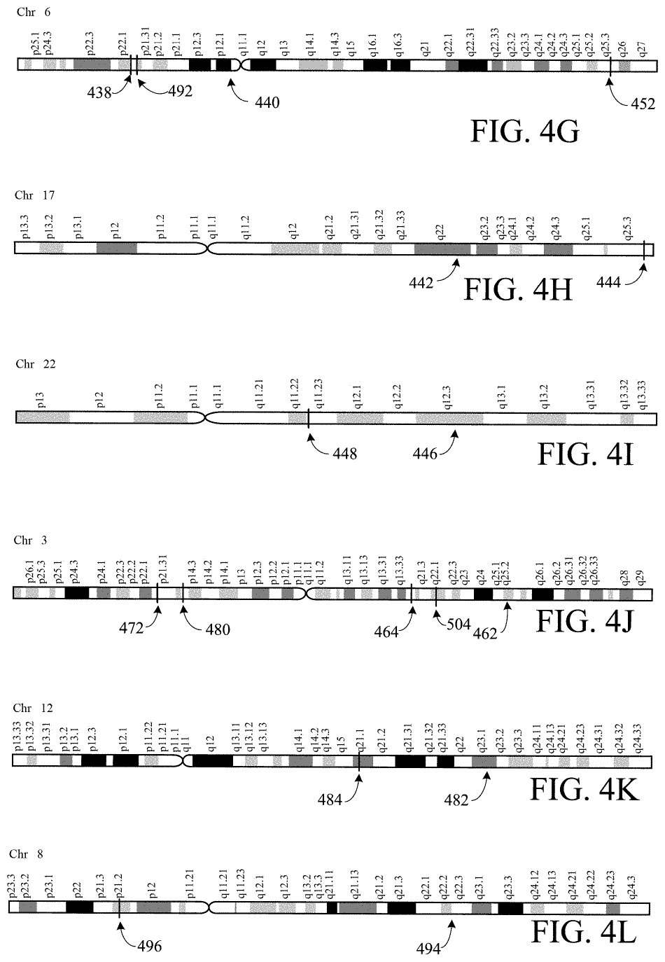

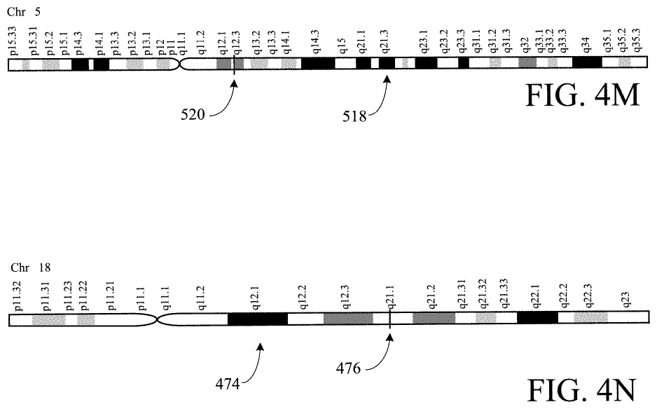

FIG. 4A-N illustrate genomic diagrams of chromosomes. Epigenetics, by definition, can alter the way in which a gene behaves based on non-sequence changing factors like methylation. If key aging gene behaviors are modified by methylation, then the methylation status of CpG sites in these genes may provide insight on the biological age of an individual.

FIG. 4A illustrates a genomic diagram 402 of chromosome 11. At location 404 is the Fibroblast growth Factor 19 (FGF19) gene. FGF family members possess broad mitogenic and cell survival activities, and are involved in a variety of biological processes including embryonic development cell growth, morphogenesis, tissue repair, tumor growth and invasion. The FGF19 protein is produced in the gut where it functions as a hormone, regulating bile acid synthesis, with effects on glucose, cholesterol, and lipid metabolism. Reduced synthesis, and blood levels, has been linked to chronic bile acid diarrhea and as well as certain metabolic diseases. FGF19 may be used in treatment of metabolic disease. FGF19 plays a role in maintaining health and metabolic homeostasis. High levels of circulating FGF19 increases energy spending and reduces fat tissue. FGF19 can have hypertrophic effect on skeletal muscle. FGF19, therefore, appears be a therapeutic target to limit aging-associated muscle loss and other diseases characterized by muscle atrophy (obesity, cancer, kidney failure). The CpG site, Cg27330757, is a probe mapping to protein coding Fibroblast growth Factor 19 (FGF19) gene.

At location 420 is the Mono-ADP Ribosylhydrolase 1 (MACROD1) gene. The protein encoded by this gene has a mono-ADP-ribose hydrolase enzymatic activity, i.e. removing ADP-ribose in proteins bearing a single ADP-ribose moiety, and is thus involved in the finetuning of ADP-ribosylation systems. ADP-ribosylation is a (reversible) post-translational protein modification controlling major cellular and biological processes, including DNA damage repair, cell proliferation and differentiation, metabolism, stress, and immune responses. MACROD1 appears to primarily be a mitochondrial protein and is highly expressed in skeletal muscle (a tissue with high mitochondrial content). It has been shown to play a role estrogen and androgen signaling and sirtuin activity. Dysregulation of MACROD1 has been associated with familial hypercholesterolemia and pathogenesis of several forms of cancer, and particular with progression of hormone-dependent cancers. The CpG site, cg15769472, is a probe mapping to the protein coding MACROD1 gene.

At location 508 is the Diacylglycerol Kinase Zeta (DGKZ) gene. The DGKZ gene coding for the diacylglycerol kinase zeta. This kinase transforms diacylglycerol (DAG) into phosphatidic acid (PA). The latter product activates mTORC1. The overall effect of mTORC1 activation is upregulation of anabolic pathways. Downregulation of mTORC1 has been shown to drastically increase lifespan. The CpG site, cg00530720, is a probe mapping to the promoter of the DGKZ gene.

At position 516 is the CD248 Molecule (CD248) gene. The CpG site, cg06419846, is a probe mapping to the CD248 gene.

FIG. 4B illustrates a genomic diagram 406 of chromosome 15. At location 408 is the WAS Protein Homolog Associated with Actin, Golgi Membranes and Microtubules Pseudogene 3 (WHAMMP3) gene. This pseudogene has been associated with Prader-Willi syndrome, a severe developmental disorder. This syndrome is caused by epigenetics defect on chromosome 15. More specifically the absence of paternally expressed imprinted genes at 15a11.2-q13, paternal deletions of this region, maternal uniparental dysomy of chromosome 15 or an imprinting defect. Multiple imprinted genes in this region contribute to the complete phenotype of Prader-Willi. The CpG site, cg04777312, is a probe mapping to the pseudogene WHAMMP3.

At location 424 is the PML gene. The phosphoprotein coded by this gene localizes to nuclear bodies where it functions as a transcription factor and tumor suppressor. Expression is cell-cycle related and it regulates the p53 response to oncogenic signals. The gene is often involved in the PML-RARA translocation between chromosomes 15 and 17, a key event in acute promyelocytic leukemia (APL). The exact role of PML-nuclear body (PML-NBs) interaction is still under further investigation. Current consensus is that PML-NBs are structures which are involved in processing cell damages and DNA-double strand break repairs. Interestingly, these PML-NBs bodies have been shown to decrease with age and their stress response also declines with age. The latter can be in a p53 dependent or independent way. PML has also been implicated in cellular senescence, particularly its induction and acts as a modulator of the Werner syndrome, a type of progeria. The CpG site, cg05697231, is a probe mapping to the south shore of a CpG island in the PML gene.

At location 456 is the ADAM Metallopeptidase with Thrombospondin Type 1 Motif 17 (ADAMTS17) gene. The CpG site, cg07394446, is a probe mapping to ADAMTS17.

At location 460 is the SMAD Family Member 6 (SMAD6) gene. The CpG site, cg07124372, is a probe mapping to SMAD6.

At location 488 is the Carbonic Anhydrase 12 (CA12) gene. The CpG site, cg10091775, is a probe mapping to CA12.

FIG. 4C illustrates a genomic diagram 410 of chromosome 2. At location 412 is the Bridging Integrator 1 (BIN1) gene. The BIN1 gene provides instructions for making a protein that is found in tissues throughout the body, where it interacts with a variety of other proteins. The BIN1 protein is involved in endocytosis as well as apoptosis, inflammation, and calcium homeostasis. The BIN1 protein may act as a tumor suppressor protein, preventing cells from growing and dividing too rapidly or in an uncontrolled way. In addition to its roles in tumor suppression and muscle development, multiple GWAS studies identified BIN1 as risk factor for late-onset Alzheimer's disease. While the exact pathogenic mechanism of BIN1 is still unknown, both high-risk variants and. DNA methylation have been suggested as mechanisms affecting BIN1 transcription and Alzheimer's Disease risk. The CpG site, cg27405400, is a probe mapping to the BIN1 gene.

At location 428 is the Protein Tyrosine Phosphatase Receptor Type N (PTPRN) gene. This gene codes for a protein receptor involved in a multitude of processes including cell growth, differentiation, mitotic cycle and oncogenic transformation. More specifically, this PTPRN plays a significant role in the signal transduction of multiple hormone pathways (neurotransmitters, insulin and pituitary hormones). PTPRN expression levels are also used as a prognostic tool for hepatocellular carcinoma (negative outcome correlation). The CpG site, cg03545227, is a probe mapping in the protein tyrosine phosphatase receptor type N (PTPRN) gene.

FIG. 4D illustrates a genomic diagram 414 of chromosome 19. At location 416 is the Kruppel-Like Factor 2 (KLF2) gene. This gene encodes for a transcription factor (zinc finger protein) found in many different cell types. Expression starts early in mammalian development and plays a role in processes ranging from adipogenesis, embryonic erythropoiesis, epithelial integrity, inflammation, and t-cell viability. Its role in inflammation is of specific interest as chronic systemic inflammation highly correlates with aging and age associated disease. The downstream effect of KLF2 expression is the downregulation of inflammation and reduction of pro-inflammatory activity of nuclear factor kappa beta (NF-kB). In humans with chronic infections and inflammatory disease such as sepsis, rheumatoid arthritis, atherosclerosis a reduction of 30 to 50% in KLF2 levels has been observed. The CpG site, cg26842024, is a probe mapping to a CpG island in the Kruppel-Like Factor 2 (KLF2) gene.

At location 512 is the Midnolin (MIDN) gene. The CpG site, cg07843568 is a probe mapping to MIDN.

FIG. 4E illustrates a genomic diagram 430 of chromosome 1. At location 432 is the Multiple EGF Like Domains 6 (MEGF6) gene. This gene plays a role in cell adhesion, motility and proliferation and is also involved in apoptotic cell phagocytosis. Mutations in this gene are associated with a predisposition to osteoporosis. The CpG site, cg23686029, is a probe mapping in the MEGF6 gene.

FIG. 4F illustrates a genomic diagram 434 of chromosome 7. At location 436 is the Kruppel-Like Factor 14 (KLF14) gene. This gene encodes a member of the Kruppel-like family of transcription factors and shows maternal monoallelic expression in a wide variety of tissues. The encoded protein functions as a transcriptional co-repressor and is induced by transforming growth factor-beta (TGF-beta) to repress TGF-beta receptor II gene expression. This gene exhibits imprinted expression from the maternal allele in embryonic and extra-embryonic tissues. Variations near this transcription factor are highly associated with coronary artery disease. The CpG site, cg08097417, is a probe mapping to the KLF14 gene.

At location 468 is the Stromal Antigen 3 (STAG3/GPC2) gene. The CpG site, cg18691434, is a probe mapping to the STAG3/GPC2 gene. At location 500 is the Huntingtin Interacting Protein 1 (HIP1) gene. The CpG site, cg13702357, is a probe mapping to the HIP1 gene. At location 524 is the DPY19L2 Pseudogene 4 (DPY19L2P4) gene. The CpG site, cg22370005, is a probe mapping to the DPY19L2P4 gene.

FIG. 4G illustrates a genomic diagram 438 of chromosome 6. At location 440 is the Protein Phosphatase 1 Regulatory Subunit 18 (PPP1R18) gene. The protein that is encoded by this gene, phosphatase-1 (PP1), plays a role in glucose metabolism in the liver by controlling the activity of phosphorylase a that breaks down glycogen to release glucose in the blood stream. Furthermore, PP1 is also involved in diverse, essential cellular processes such as cell cycle progression, protein synthesis, muscle contraction, carbohydrate metabolism, transcription and neuronal signaling. In Alzheimer's disease, expression of PP1 is significantly reduced in both white and grey matter. The CpG site, cg23197007, is a probe that maps to the S-shore of the PPP1R18 gene.

At location 452 is the TMEM181 gene. cg02447229 is a probe mapping to the TMEM181 gene. At location 492 is the Zinc Finger and BTB Domain Containing 12 (ZBTB12) gene. The CpG site, cg06540876, is a probe mapping to the ZBTB12 gene.

FIG. 4H illustrates a genomic diagram 442 of chromosome 17. At location 444 is the dicarbonyl and L-xylulose reductase (DCXR) gene. One of its functions is to perform a chemical reaction that converts a sugar called L-xylulose to a molecule called xylitol. This reaction is one step in a process by which the body can use sugars for energy. There are two versions of L-xylulose reductase in the body, known as the major isoform and the minor isoform. The DCXR gene provides instructions for making the major isoform, which converts L-xylulose more efficiently than the minor isoform. It is unclear if the minor isoform is produced from the DCXR gene or another gene. Another function of the DCXR protein is to break down toxic compounds called alpha-dicarbonyl compounds. The DCXR protein is also one of several proteins that get attached to the surface of sperm cells as they mature. DCXR is involved in the interaction of a sperm cell with an egg cell during fertilization. The CpG site, cg07073120, is a probe that maps to the promotor region of the DCXR gene.

FIG. 4I illustrates a genomic diagram 446 of chromosome 22. At location 448 is the BCR Activator of RhoGEF and GTPase (BCR) gene. The CpG site, cg04028010, is a probe mapping to the BCR gene.

FIG. 4J illustrates a genomic diagram 462 of chromosome 3. At location 464 is the Myosin Light Chain Kinase (MYLK) gene. This gene codes for a myosin light chain kinase (MLCK), which is a calcium/calmodulin dependent enzyme active in smooth muscle tissue (involuntary muscle). It phosphorylates myosin light chains to facilitate their interaction with actin to produce muscle contraction. A second function of the MLCK protein is regulation of the epithelial tight junction. These are the gaps between the epithelial cells and their size is of major biological importance as this determines the selective permeability of the epithelial barrier. This selectivity allows absorption and secretion of nutrients and metabolites while protecting the sterile tissues against pathogens. A secondary promotor drives the expression of a second gene transcript: telokin. This small protein is identical to the c-terminus of the MYLK protein and helps stabilize unphosphorylated myosin filaments. Abnormal expression of MYLK have been observed in many inflammatory diseases such as, pancreatitis, asthma, inflammatory bowel disease.

At location 472 is the Transglutaminase 4 (TGM4) gene. The CpG site, cg12112234, is a probe mapping to the TGM4 gene.

At location 480 is the Scm Like With Four Mbt Domains 1 (SFMBT1) gene. The CpG site, cg03607117, is a probe mapping to the SFMBT1 gene.

At location 504 is Nudix Hydrolase 16 (NUDT16P) gene. The CpG site, cg22575379, is a probe mapping to the NUDT16P gene.

FIG. 4K illustrates a genomic diagram 482 of chromosome 12. At location 484 is the Thyrotropin Releasing Hormone Degrading Enzyme (LOC283392/TRHDE) gene. The CpG site, cg13663218, is a probe mapping to the LOC283392/TRHDE gene.

FIG. 4L illustrates a genomic diagram 496 of chromosome 8. At location 496 is the Neurofilament Medium Chain (NEFM) gene. The CpG site, cg07502389, is a probe mapping to the NEFM gene.

FIG. 4M illustrates a genomic diagram 518 of chromosome 5. At location 520 is the Ring Finger Protein 180 (RNF180) gene. The RNF180 gene codes for RING-Type E3 Ubiquitin Transferase. RING-type E3s are implicated as tumor suppressors, oncogenes, and mediators of endocytosis, and play critical roles in complex multi-step processes such as DNA repair and activation of NF-κB a master regulator of inflammation. RING-type E3s and their substrates are implicated in a wide variety of human diseases ranging from viral infections to neurodegenerative disorders to cancer. The CpG site, cg23008153, is a probe mapping to the N-shore of the RNF180 gene.

FIG. 4N illustrates a genomic diagram 474 of chromosome 18. At location 476 is the SMAD Family Member 2 (SMAD2) gene. The CpG site, cg17243289, is a probe mapping to the SMAD2 gene.

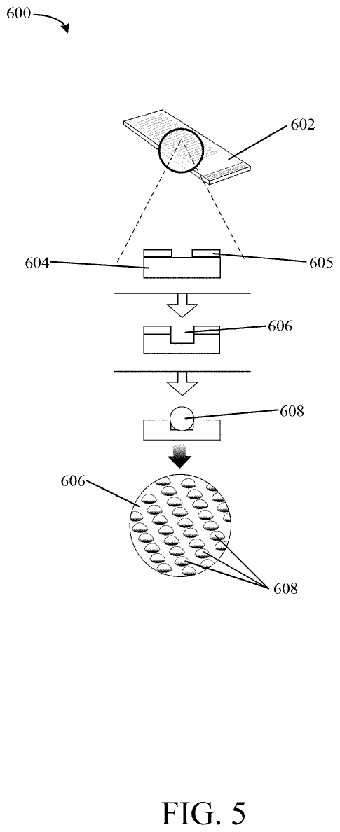

FIG. 5 is a diagram showing an example array chip 602 used to detect methylation. Array chip 602 includes a silicon wafer 604 which is coated with a photo-resistant material 605. Array chip 602 include a plurality of microwells 606 disposed along its surface. Microwells 606 are not covered by the photo-resistant material 605 and extend depth-wise into the silicon wafer 604. Each microwell 606 houses one or more beads 608. Beads 608 are coated with multiple copies of an oligonucleotide probe targeting a specific location in the genome. As sample DNA fragments pass over beads 608, each probe binds to a complementary sequence in the sample DNA, stopping proximate the location of interest. Thus, specific locations in the DNA sample can be targeted for analysis. For instance, locations corresponding to CpG sites can be targeted for methylation state analysis. Specific beads 608 can be utilized to complete a methylation state analysis.

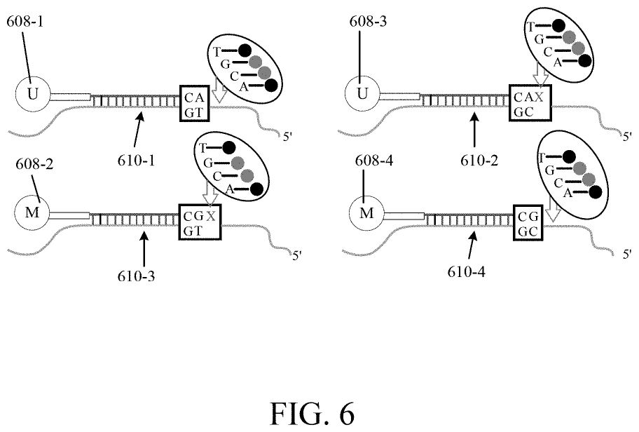

FIG. 6 is a diagram showing example beads 608 that respond to methylated and unmethylated CpG sites. Before DNA is exposed to the methylation analysis beads 608, the DNA is treated with sodium bisulfite. Sodium bisulfite converts cytosine into uracil, but leaves methylated cytosine (e.g., 5-methylcytosine) unaffected. The array chip 602 interrogates these chemically differentiated locations using two site-specific probes, one bead type (U) (beads 608-1, 608-3) presents probes that are designed to match to an unmethylated site; the second bead type (M) (beads 608-2, 608-4) matches a methylated state. Single-base extension of the probes incorporates a labeled dideoxynucleotides (ddNTP), which is subsequently stained with a fluorescent reagent. The level of methylation for the interrogated location can be determined by calculating the ratio of the fluorescent signals from the methylated vs. unmethylated sites.

On the left side of this figure, the locus of interest is unmethylated. It matches perfectly with unmethylated bead probe 608-1, enabling single-base extension and detection. The unmethylated locus has a single-base mismatch to the methylated bead probe 608-2, inhibiting extension that results in a low signal on the array. If the CpG locus of interest is methylated, the reverse occurs: the methylated bead 608-4 type will display a signal, and the unmethylated bead 608-3 type will show a low signal on the array. If the locus has an intermediate methylation state, both probes will match the target site and will be extended. Methylation status of the CpG site is determined by a β-value calculation, which is the ratio of the fluorescent signals from the methylated beads to the total locus intensity. The array chip containing the beads 608 can be read by an array scanning device, such as the iScan™ System provided by Illumina or the NexSeq™ 550 System provided by Illumina.

FIGS. 5-6 disclose one system to detect methylation. The present disclosure also explicitly contemplates using other methods to detect the methylation status of CpG sites.

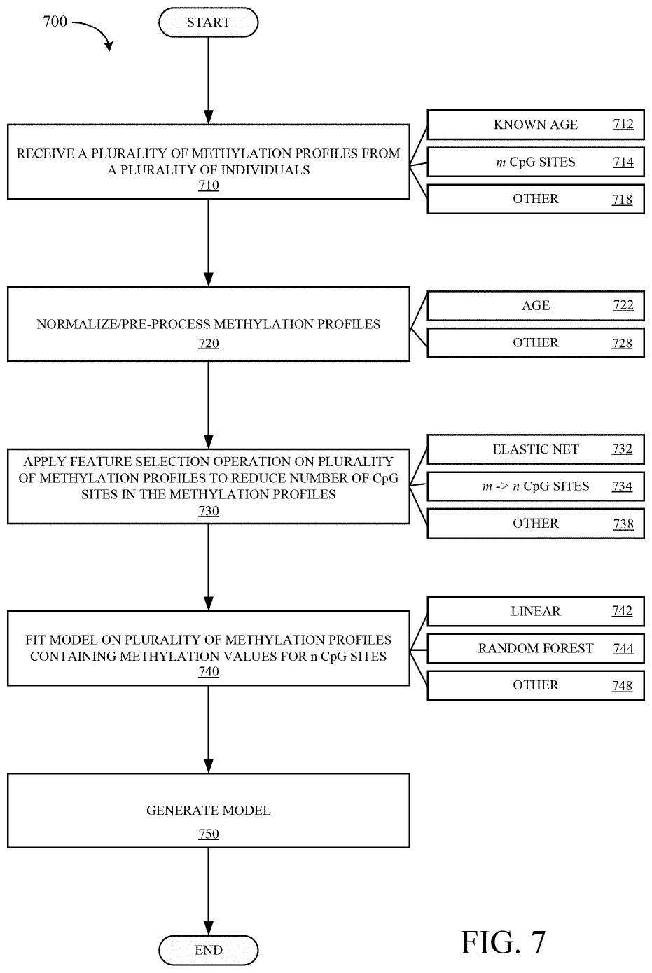

FIG. 7 illustrates a flow diagram showing an example operation 700 of training an epigenetic age predicting model. Operation 700 begins at block 710 where a plurality of methylation profiles from a plurality of individuals are received. A methylation profile contains methylation values for a number of CpG sites of the individual. Methylation values can be in a number of different formats. An example format is a decimal between zero and one (β-value), where zero is fully unmethylated and one is fully methylated. These methylation profiles for the individuals can be derived from an array described above with respect to FIGS. 5-6.

As indicated by block 712, the plurality of individuals' ages are known. This known age can be used to generate the model. For example, the plurality of methylation values in each profile can be used as an input vector and the known age is the scalar output value.

As indicated by block 714, the number of CpG sites in each of the plurality of methylation profiles is m. The quantity m can be a variety of different numbers. For example, m can correspond to a resolution of the methylation analysis method used on the plurality of individuals. For instance, Illumina provides methylation microarrays that detect ˜850,000 CpG sites (Infinium methylation EPIC array) or, for instance, Illumina provides methylation sequencing for 3.3 million or 36 million CpG sites.

Operation 700 proceeds at block 720 where the plurality of methylation profiles received in block 710 are normalized or otherwise pre-processed. As indicated by block 722 the methylation profiles can be normalized based on age. For instance, a specific age range may be overrepresented in the plurality of profiles, therefore the profiles from this age range may need to be weighted less or their chance of being sampled made less likely. In the alternate, a specific age range may be underrepresented in the plurality of profiles, therefore the profiles from this age range may need to be weighted higher or their chance of being sampled made more likely.

As indicated by block 724, the plurality of methylation profiles may be curated based on a quality metric. For example, methylation profiles that have a quality metric lower than a threshold are discarded or weighted less than methylation profiles of a higher quality metric. In some examples, the quality metric is indicative of the accuracy of the methylation process. In some examples, the quality metric is indicative of the quality of the sample used to generate the particular methylation profile. In some examples, the quality metric is indicative of the quality of the test used to generate the particular methylation profile. In other examples, the quality metric may be indicative of other factors relating the particular methylation profile. As indicated by block 728, the plurality of methylation profiles can be normalized or pre-processed in other ways as well.

Operation 700 proceeds at block 730 where a feature selection operation is applied on the plurality of methylation profiles. In some examples, the feature selection is applied on the plurality of methylation profiles after they are pre-processed. In some examples, the feature selection operation is applied on the plurality of methylation profiles received in block 710.

As indicated by block 732, the feature selection operation applied on the plurality of methylation includes elastic net regression. Elastic net regression combines L1 penalties from lasso regression and L2 penalties from ridge regression to reduce the number of CpG sites.

As indicated by block 734, the feature selection applied reduces the number of applicable CpG sites from m to n sites. This reduction can balance dimensionality when there are small number of methylation profiles, but each profile has a large amount of CpG site methylation values. In some examples, m is greater than 100,000. In some examples, m is greater than 400,000. In some examples, m is greater than 800,000. In some examples, n is less than 200. In some examples, n is less than 100. In some examples, n is less than 50.

Operation 700 proceeds at block 740 where a model is fit on the plurality of methylation profiles. In some implementations, the model is fit on the plurality of methylation profiles, but only considers the CpG sites in the subset of n CpG sites. In some implementations, the model is fit on all m CpG sites. As indicated by block 742, the model can include a linear regression model. As indicated by block 744, the model can include a random forest model. As indicated by block 748, the model can include a different type of model as well.

Operation 700 proceeds at block 750 where the model is generated. In some examples, the model is generated as one or more files that can be imported by other systems which can further train the model or use the model for predictions.

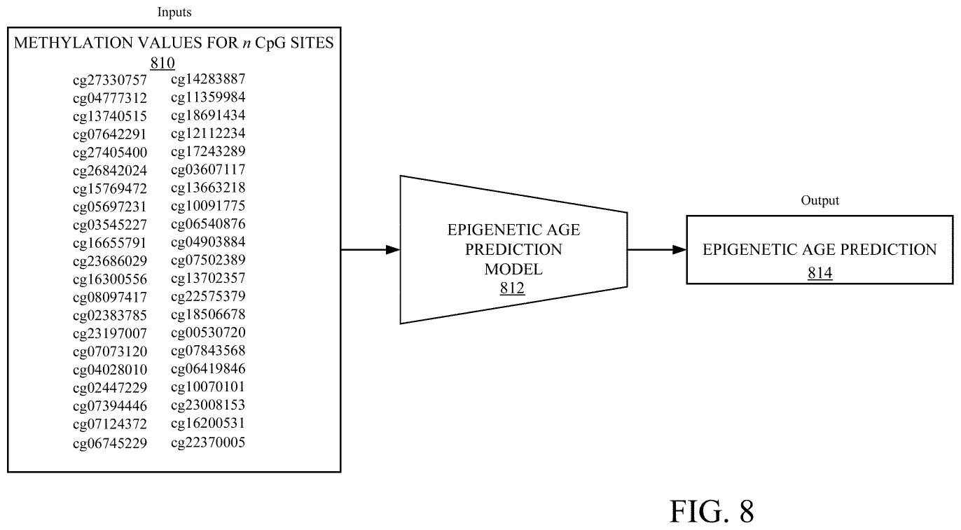

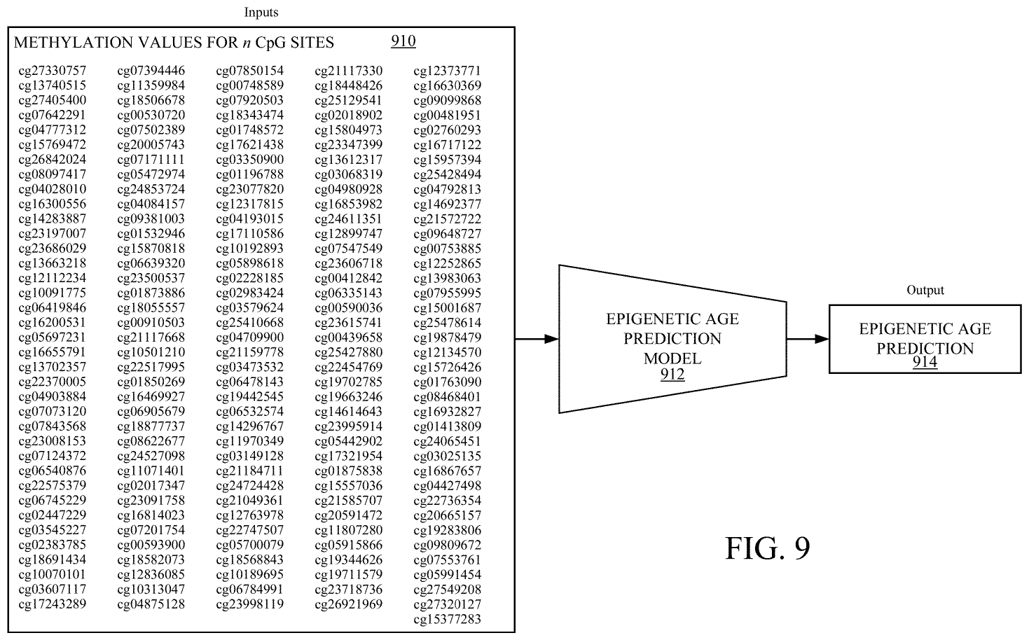

FIGS. 8-9 are flow diagrams showing example operations of predicting an epigenetic age. Operation 800 begins at block 810 where the input values are received. As shown, there are 42 input values corresponding to the methylation values at 42 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model). In one implementation, the input values are derived from a methylation analysis on a sample of human blood.

At block 812, the inputs are input into the epigenetic age prediction model. As noted above this model could include a linear regression model (e.g., where each input forms part of a linear expression), random forest model (e.g., where each input forms part of one or more decision trees), or some other type of model. At block 814, the model outputs an epigenetic age prediction. In some implementations, the model also outputs a confidence score.

Operation 900 begins at block 910 where the input values are received. As shown, there are 186 input values corresponding to the methylation values at 186 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model). In one implementation, the input values are derived from a methylation analysis on a sample of human blood.

At block 912, the inputs are input into the epigenetic age prediction model. As noted above this model could include a linear regression model (e.g., where each input forms part of a linear expression), random forest model (e.g., where each input forms part of one or more decision trees), or some other type of model. At block 914, the model outputs an epigenetic age prediction. In some implementations, the model also outputs a confidence score.

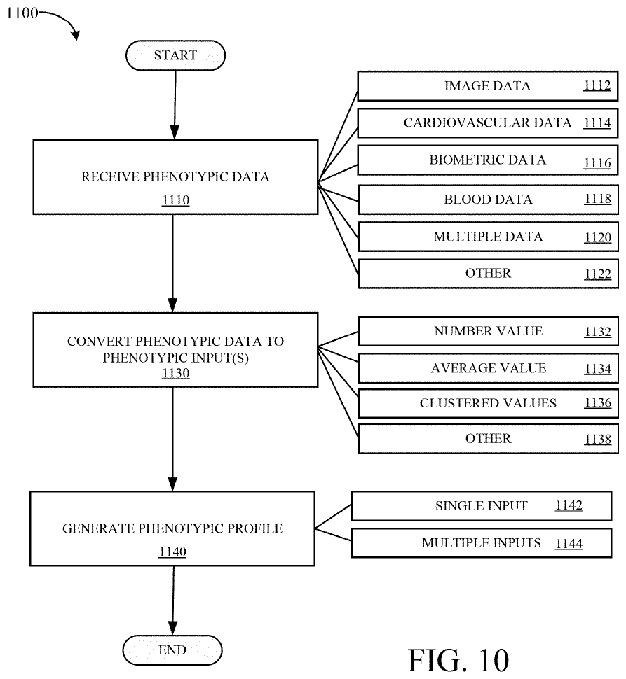

FIG. 10 illustrates a flow diagram showing an example operation of generating a phenotypic profile. A phenotypic profile can include one or more sets of observable phenotypes of an individual. Phenotypes can include, for example, physical characteristics observable on the skin, such as wrinkles, blemishes, color, growths, etc. In addition to observable characteristics, a phenotypic profile can also include biochemical and/or physiological conditions relative to the individual, such as cardiovascular conditions, biometrics, blood conditions, etc. The phenotypic profile of an individual can provide one or more additional inputs indicative of the individual's epigenetic age. For instance, an individual can have a determined epigenetic age by the methods described above with respect to FIGS. 8-9 relative to the degree of methylation at the plurality of CpG sites. However, when considering one or more phenotypic characteristics of the individual, the epigenetic age can be adjusted relative to the phenotypic characteristics in combination with the individual's methylation values.

Operation 1100 begins at block 1110 where one or more sources of phenotypic data is received. Receiving phenotypic data can include, for example, receiving image data 1112. In one example, image data 1112 can be contained within an image of an individual (e.g., a facial image). The image received can be produced in a number of different formats. For example, the image can be an image taken by a camera. In another example, the image can be taken by a camera on a computer, such as a webcam. In another example, the received image can be produced by a camera on a mobile device (e.g., a smartphone). Additionally, it is expressly contemplated that the image can be produced by other manners as well. The received image can be in a number of different formats. For example, the image can be a JPEG image, PNG image, PDF, or any other suitable image format. Image data 1112 includes data relative to the individual's epigenetic age. For example, image data 1112 can include data relating to the individual's skin (e.g., wrinkles, blemishes, growths, marks, color, etc.). Additionally, image data 1112 can include data relating to other phenotypic characteristics of the individual as well, such as data relating to facial features (e.g., eyes, nose, mouth, etc.). Image data 1112 can be utilized to generate a phenotypic profile of the individual, which can provide further information pertaining to epigenetic age.

Receiving phenotypic data can also include, for example, receiving cardiovascular data 1114. Cardiovascular data 1114 can include, for example, heart rate data, such as heart rate variability (HRV), maximum heart rate (Max. HR), minimum heart rate (Min. HR), heart rate range, resting heart rate, etc. In one example, cardiovascular data 1114 includes a plurality of cardiovascular data types. However, in another example, cardiovascular data 1114 can include only one type of cardiovascular data.

Receiving phenotypic data 1110 can also include receiving biometric data 1116. Biometric data includes body measurements and/or calculations relative to the physical characteristics of the individual. For example, biometric data 1116 can include the individual's basal metabolic rate (BMR). In other examples, biometric data 1116 can include the individual's waist circumference, body mass index (BMI), fat percentage, muscle percentage, muscle-to-fat ratio, lean muscle mass, visceral fat mass, bone mass, oxygen consumption rate (VO₂ max), etc. In one example, biometric data 1116 includes one type of biometric data (e.g., BMR). However, in other examples, biometric data 1116 can include multiple biometric data types, as indicated above.

In another example, receiving phenotypic data can also include receiving blood data 1118. Blood data 1118 can be obtained by measuring an individual's blood characteristics using conventional methods. For example, blood pressure of the individual can be obtained using a blood pressure monitor. Additionally, blood data 1118 can be received by a blood sample of the individual. The blood sample can be obtained in a number of ways. For instance, the blood sample can be obtained by a blood extraction device (e.g., a fingerstick). In another example, the blood sample can be obtained by use of a needle and syringe. Additionally, it is expressly contemplated that the blood sample can be obtained in other manners as well.

The blood sample can be processed to obtain blood data 1118. Processing the blood sample can include, for example, a complete blood count (CBC) or blood panel. Additionally, processing the blood can include other conventional means of blood analysis as well, such as machine analysis, centrifugation, etc. The blood data 1118 obtained from the blood sample may include a number of different data types. For example, blood data 1118 can include blood oxygen levels, lipid profiles (e.g., total cholesterol, low-density lipoproteins, high-density lipoproteins, triglycerides, etc.), sugar profiles (e.g., glucose, hemoglobin A1C, etc.), hormone levels (e.g., cortisol, testosterone, estrogen, etc.), and other parameters. In one example, blood data 1118 includes one type of blood data such as, for example, blood oxygen level. However, in other examples, blood data 1118 can include multiple blood data types, as indicated above.

As indicated by block 1120, receiving phenotypic data 1110 can include receiving multiple data types from multiple sources. For instance, image data 1112 can be received in addition to, for example, cardiovascular data 1114. In another example, cardiovascular data 1114 can be received in addition to biometric data 1116. In another example, data is received from all the above-indicated data sources. However, in another example, only one data type can be received as well. For example, the phenotypic data received may only include image data 1112. Additionally, it is expressly contemplated that other data types can be received as well, as indicated by block 1122.

Operation 1100 proceeds at block 1130, where the phenotypic data is converted to one or more phenotypic inputs. Converting phenotypic data to phenotypic inputs includes converting the phenotypic data to a measurable value that can be inputted into an epigenetic age prediction model to provide an individual's modified epigenetic age. For example, converting the phenotypic data can include obtaining phenotypic inputs from image data indicative of one or more phenotypic characteristics of the individual. The phenotypic data can be, for example, converted to an input in a format of a number value, as indicated by block 1132. For instance, in the case where the phenotypic data includes image data, the image data can be converted to an input of a decimal between zero and one (β-value), where zero indicates that there are no notable phenotypic markings (e.g., facial wrinkles) and one indicates that there is a high level of phenotypic markings. Additionally, image data can be converted to a different measurable phenotypic input as well, such as a different number value.

As indicated by block 1134, converting the phenotypic data to one or more phenotypic inputs can include averaging the phenotypic data. Averaging can occur for any of the phenotypic data examples set forth above with respect to blocks 1112-1118. For example, if multiple data sets of cardiovascular data are obtained relative to an individual (e.g., a plurality of heart rate values), the data can be averaged to generate an average value of the cardiovascular data. In another example, multiple data sets of biometric data can be obtained relative to an individual (e.g., a plurality of VO₂ max measurements), where the data is averaged to generate an average value of the biometric data.

As indicated by block 1136, converting the phenotypic data to one or more phenotypic inputs can also include clustering values of relevant data types together. For example, data relating to an individual's total cholesterol, blood sugar profiles, and blood pressure can be organized as a set of blood inputs. In another example, data relating to an individual's BMR, BMI, and bone mass can be organized as a set of biometric inputs. In another example, physical characteristics present in an image, such as facial wrinkles, blemishes, and growths, can be organized as a set of image inputs. Additionally, it is expressly contemplated that other ways of converting phenotypic data to phenotypic inputs can be utilized as well, as indicated by block 1138.

Operation 1100 proceeds at block 1140 where the phenotypic profile of the individual is generated. A phenotypic profile contains one or more phenotypic inputs relative to the individual. For example, the phenotypic profile can include one or more sets of observable phenotypes of an individual. In addition to observable characteristics, the phenotypic profile can also include biochemical and/or physiological conditions relative to the individual, as indicated above. In this way, the phenotypic profile can provide one or more additional inputs indicative of the individual's epigenetic age. As indicated in block 1142, the phenotypic profile can comprise a single phenotypic input. For example, the phenotypic profile can include phenotypic inputs indicative of the obtained image data. In another example, the phenotypic profile can include phenotypic inputs indicative of the obtained biometric data. Additionally, as indicated by block 1144, the phenotypic profile can also include multiple phenotypic inputs. For example, the phenotypic profile can include one or more inputs obtained from image data 1112 in combination with one or more inputs obtained from cardiovascular data 1114. In another example, the phenotypic profile can include a combination of all the data types set forth above with respect to blocks 1112-1118.

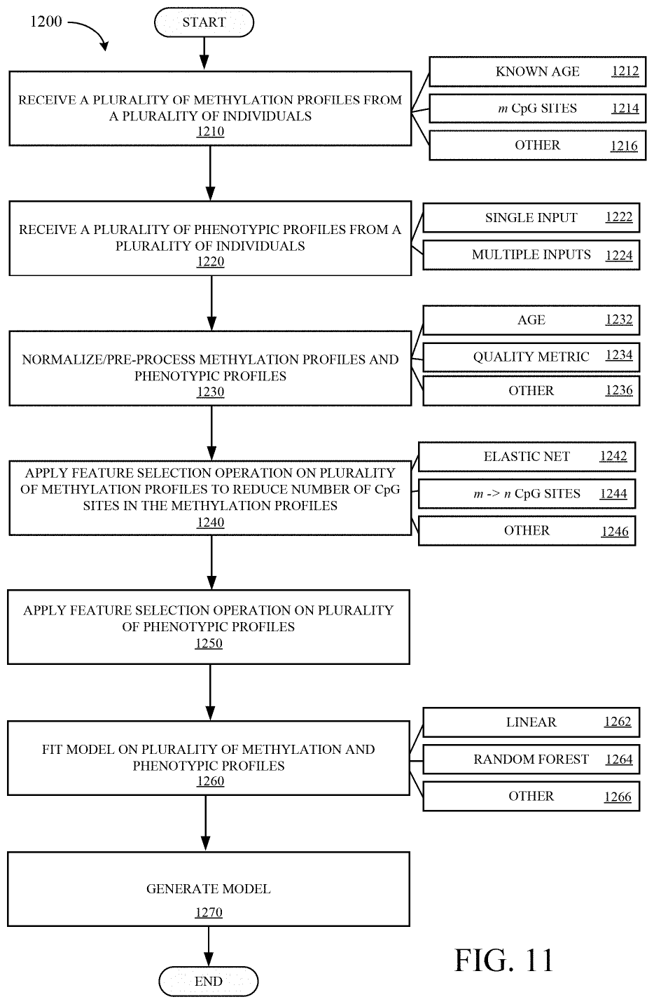

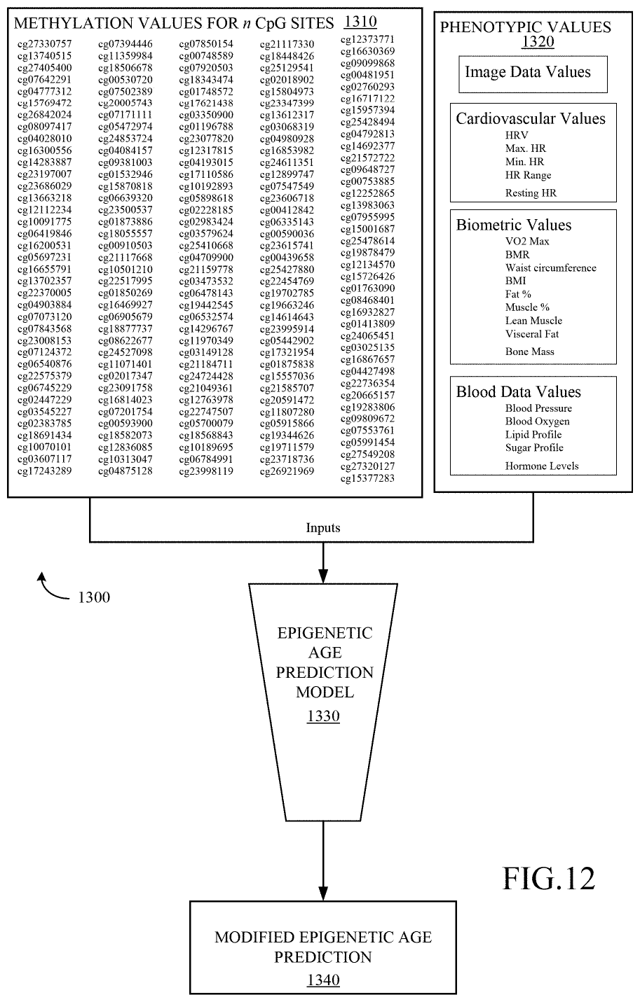

FIG. 11 illustrates a flow diagram showing an example operation of training a model. As shown, operation 1200 bears some similarities to operation 700 described above with respect to FIG. 7. However, operation 1200 additionally utilizes the incorporation of a plurality of phenotypic profiles from a plurality of individuals. Operation 1200 begins at block 1210 where a plurality of methylation profiles from a plurality of individuals are received. A methylation profile contains methylation values for a number of CpG sites of the individual. Methylation values can be in a number of different formats. An example format is a decimal between zero and one (β-value), where zero is fully unmethylated and one is fully methylated. These methylation profiles for the individuals can be derived from an array described above with respect to FIGS. 5-6.

As indicated by block 1212, the plurality of individuals' ages are known. This known age can be used to generate the model. For example, the plurality of methylation values in each profile can be used as an input vector and the known age is the scalar output value. Additionally, as indicated by block 1214, the number of CpG sites in each of the plurality of methylation profiles is m. The quantity m can be a variety of different numbers. Additionally, the methylation profiles of each individual of the plurality of individuals can include other information as well, as indicated by block 1216.

Operation 1200 proceeds at block 1220 where a plurality of phenotypic profiles from a plurality of individuals are received. The received phenotypic profiles can be, for example, derived from the same individuals where the plurality of methylation profiles were derived above with respect to block 1210. However, in another example, the plurality of phenotypic profiles can be derived from a different plurality of individuals. A phenotypic profile contains one or more phenotypic inputs relative to an individual. Phenotypic inputs can be in a number of different formats, as described above. These phenotypic profiles for the individuals can be derived from the method described above with respect to FIG. 10. As indicated by block 1222, a phenotypic profile can contain a single phenotypic input. For example, the phenotypic profile can contain phenotypic inputs from image data obtained by the individual indicative of physical characteristics (e.g., wrinkles, blemishes, marks, etc.). In another example, the phenotypic profile can include phenotypic inputs from biometric data obtained by the individual. Additionally, as indicated by block 1224, a phenotypic profile can include multiple phenotypic inputs as well. For example, a phenotypic profile can include inputs from image data in combination with inputs from cardiovascular data. In another example, a phenotypic profile can include inputs from image data, cardiovascular data, biometric data, and blood data. Additionally, it is expressly contemplated that other phenotypic combinations can be included in a phenotypic profile as well.

Operation 1200 proceeds at block 1230 where the plurality of methylation profiles received in block 1210 and the plurality of phenotypic profiles received in block 1220 are normalized or otherwise pre-processed. As indicated by block 1232, the methylation profiles and phenotypic profiles can be normalized based on age. For instance, a specific age range may be overrepresented in the plurality of profiles, therefore the profiles from this age range may need to be weighted less or their chance of being sampled made less likely. In the alternate, a specific age range may be underrepresented in the plurality of profiles, therefore the profiles from this age range may need to be weighted higher or their chance of being sampled made more likely.

As indicated by block 1234, the plurality of methylation profiles and phenotypic profiles may be curated based on a quality metric. For example, phenotypic profiles that have a quality metric lower than a threshold are discarded or weighted less than the profiles of a higher quality metric. In some examples, the quality metric is indicative of the quality of the data source and obtained phenotypic inputs. For instance, if a received image containing image data is of a low resolution and/or quality, phenotypic characteristics can be difficult to determine in the image, and consequently the resulting image data can be discarded or weighted less. In another example, the quality metric is indicative of the quality of the sample used to generate the phenotypic inputs. For instance, in the case of a blood sample used to produce blood data, the blood sample can be of low quality due to error in acquiring the sample. In such a case, the resulting phenotypic data received from the blood sample may be discarded or weighted differently. Additionally, in other examples, the quality metric may be indicative of other factors relating to the particular phenotypic profile. As indicated by block 1236, the plurality of methylation profiles and/or phenotypic profiles can be normalized or pre-processed in other ways as well.

Operation 1200 proceeds at block 1240 where a feature selection operation is applied on the plurality of methylation profiles. In some examples, the feature selection is applied on the plurality of methylation profiles after they are pre-processed. In some examples, the feature selection operation is applied on the plurality of methylation profiles received in block 1210.

As indicated by block 1242, the feature selection operation applied on the plurality of methylation includes elastic net regression. Elastic net regression combines L1 penalties from lasso regression and L2 penalties from ridge regression to reduce the number of CpG sites. Additionally, as indicated by block 1244, the feature selection applied reduces the number of applicable CpG sites from m to n sites. This reduction can balance dimensionality when there are small number of methylation profiles, but each profile has a large amount of CpG site methylation values. As indicated by block 1246, other feature selection operations can be applied as well.

Operation 1200 proceeds at block 1250 where a feature selection operation can be applied to the plurality of phenotypic profiles. In some examples, the feature selection is applied on the plurality of phenotypic profiles after they are pre-processed. However, in other examples, the feature selection operation is applied on the plurality of phenotypic profiles received in block 1220. The feature selection operation applied on the plurality of phenotypic profiles can be the same as or similar to the operation applied above with respect to block 1240. For example, elastic net regression can be applied to reduce the number of phenotypic inputs used to generate the model. In another example, a different feature selection operation can be applied.

Operation 1200 proceeds at block 1260 where a model is fit on the plurality of methylation profiles. In some implementations, the model is fit on the plurality of profiles, but only considers the CpG sites in the subset of n CpG sites. In some implementations, the model is fit on all m CpG sites. Additionally, in some implementations, the model is fit on the plurality of phenotypic profiles, but only considers a specific phenotypic input (e.g., image data inputs). In other implementations, the model is fit on all phenotypic inputs. As indicated by block 1262, the model can include a linear regression model. As indicated by block 1264, the model can include a random forest model. As indicated by block 1266, the model can include a different type of model as well.

Operation 1200 proceeds at block 1270 where the model is generated. In some examples, the model is generated as one or more files that can be imported by other systems which can further train the model or use the model for predictions.

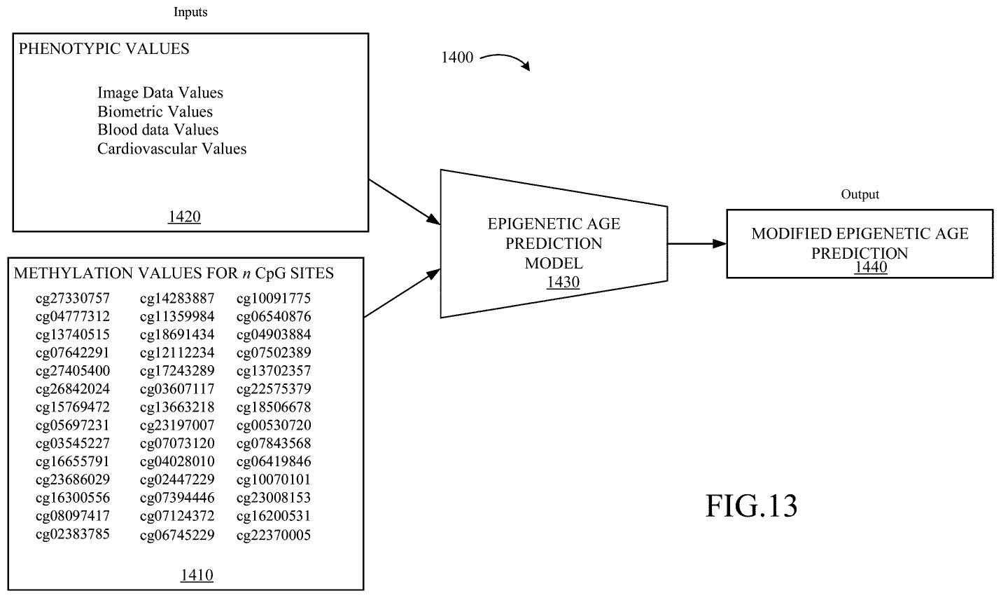

FIGS. 12-14 illustrate flow diagrams showing example operations of predicting a modified epigenetic age. Operation 1300 begins at block 1310 where methylation input values are received. As shown, there are 186 input values corresponding to the methylation values at 186 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model).

As shown at block 1320, a plurality of phenotypic input values are also received. As shown, there are four input types with corresponding subtypes for each type. The four input types correspond to image data values, cardiovascular values, biometric values, and blood data values. The four input types shown can be determined by the method described above with respect to FIG. 10. The subtypes for cardiovascular value inputs can include, for example, HRV, max. HR, min. HR, heart rate range, and resting heart rate, as shown. The subtypes for biometric value inputs can include, for example, VO₂ max, BMR, waist circumference, BMI, fat percentage, muscle percentage, lean muscle mass, visceral fat mass, and bone mass, as shown. The subtypes for blood data value inputs can include, for example, blood pressure, blood oxygen level, lipid profiles, sugar profiles, and hormone levels, as shown. In some implementations, only a subset of the shown input types and subtypes are used as inputs. However, in other implementations, all of the types and subtypes can be used as inputs.

At block 1330, the inputs are input into the epigenetic age prediction model. As noted above, this model could include a linear regression model (e.g., where each input forms part of a linear expression), random forest model (e.g., where each input forms part of one or more decision trees), or some other type of model. The model used may be, for example, the model described above with respect to FIG. 11. At block 1340, the model outputs a modified epigenetic age prediction. The modified epigenetic age prediction has some similarities to the outputted epigenetic age prediction described above with respect to FIGS. 8-9 but is modified to account for the one or more phenotypic inputs. In some examples, the modified epigenetic age prediction may be higher than the original epigenetic age prediction. In another example, the modified epigenetic age prediction may be a lower value than the original epigenetic age prediction. In another example, the modified epigenetic age prediction may be substantially the same as the original epigenetic age prediction. In some implementations, the model also outputs a confidence score.

Operation 1400 begins at block 1410 where the methylation input values are received. As shown, there are 42 input values corresponding to the methylation values at 42 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model).

As shown at block 1420, a plurality of phenotypic input values are also received. As shown, there are four input types corresponding to image data values, cardiovascular values, biometric values, and blood data values. The four input types shown may be determined by the method described above with respect to FIG. 10. Additionally, each of the four input types can include associated input subtypes. The subtypes can be, for example, the subtypes described above and shown in FIG. 12. In some implementations, only a subset of the shown input types and subtypes are used as inputs. However, in other implementations, all of the types and subtypes can be used as inputs.

At block 1430, the inputs are input into the epigenetic age prediction model. As noted above, this model could include a linear regression model (e.g., where each input forms part of a linear expression), random forest model (e.g., where each input forms part of one or more decision trees), or some other type of model. The model used may be, for example, the model described above with respect to FIG. 11. At block 1440, the model outputs a modified epigenetic age prediction. In some examples, the modified epigenetic age prediction can be higher than the original epigenetic age prediction. In another example, the modified epigenetic age prediction can be a lower value than the original epigenetic age prediction. In another example, the modified epigenetic age prediction can be substantially the same as the original epigenetic age prediction. In some implementations, the model also outputs a confidence score.

Operation 1500 begins at block 1510 where the methylation input values are received. In one example, the methylation input values received at block 1510 are 42 input values corresponding to the methylation values at 42 different CpG cites. In another example, the methylation input values received are 186 input values corresponding to the methylation values at 186 different CpG sites.

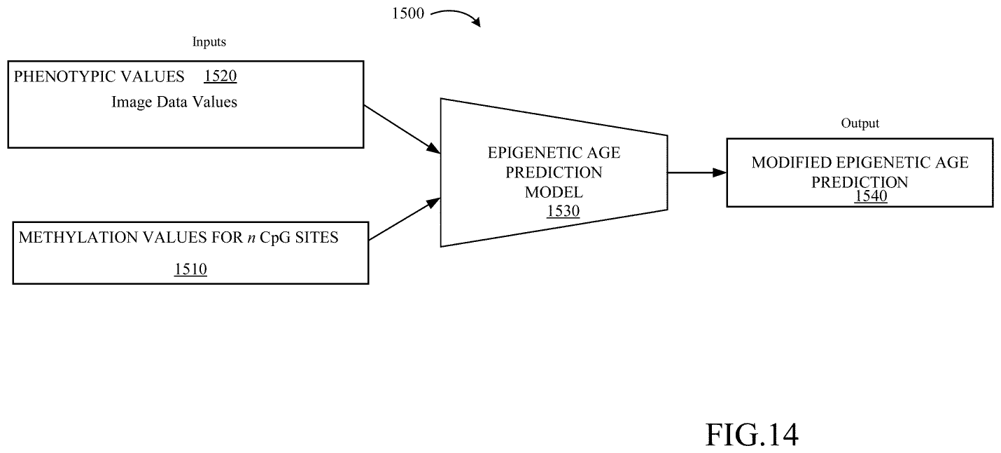

As shown at block 1520, only one phenotypic input value type is received. Specifically, as shown there is one phenotypic input type corresponding to image data. The input can be generated by the method described above with respect to FIG. 10. In this way, a single input type can be used in combination with the methylation value inputs to produce a modified epigenetic age prediction. Additionally, while image data values are shown in FIG. 14 as the only input type, it is expressly contemplated that other input types can be used in lieu of image data values as the single input type. For example, biometric data values can be used as the input. In another example, blood data values can be used as the input. In another example, cardiovascular data values can be used as the input.

At block 1530, the inputs are input into the epigenetic age prediction model. The model used may be, for example, the model described above with respect to FIG. 11. At block 1540, the model outputs a modified epigenetic age prediction based on the methylation values in combination with the phenotypic values. In some examples, the modified epigenetic age prediction can be higher than the original epigenetic age prediction. In another example, the modified epigenetic age prediction can be a lower value than the original epigenetic age prediction. In another example, the modified epigenetic age prediction can be substantially the same as the original epigenetic age prediction. In some implementations, the model also outputs a confidence score. As shown in FIG. 14, a modified epigenetic age can therefore be output using only a single phenotypic input type, if desired.

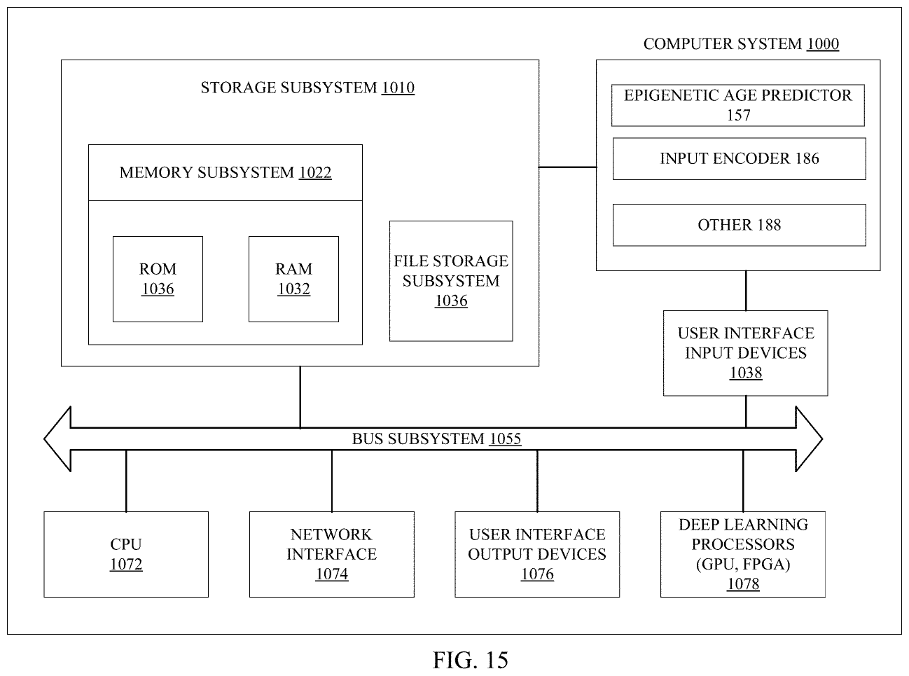

FIG. 15 is a computer system 1000 that can be used to implement the convolution-based base calling and the compact convolution-based base calling disclosed herein. Computer system 1000 includes at least one central processing unit (CPU) 1072 that communicates with a number of peripheral devices via bus subsystem 1055. These peripheral devices can include a storage subsystem 1010 including, for example, memory devices and a file storage subsystem 1036, user interface input devices 1038, user interface output devices 1076, and a network interface subsystem 1074. The input and output devices allow user interaction with computer system 1000. Network interface subsystem 1074 provides an interface to outside networks, including an interface to corresponding interface devices in other computer systems.

In one implementation, the epigenetic age predictor 157 is communicably linked to the storage subsystem 1010 and the user interface input devices 1038. Epigenetic age predictor 157 can include one or more models that receive a plurality of inputs and output an epigenetic age. In some examples, epigenetic age predictor 157 also outputs a confidence score. In one implementation, input encoder 186 pre-processes and/or normalizes inputs before they are fed into a model of epigenetic age predictor.

User interface input devices 1038 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1000.

User interface output devices 1076 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1000 to the user or to another machine or computer system.

Storage subsystem 1010 stores programming and data constructs that provide the functionality of some or all of the modules and methods described herein. These software modules are generally executed by deep learning processors 1078.

Deep learning processors 1078 can include graphics processing units (GPUs), field-programmable gate arrays (FPGAs), application-specific integrated circuits (ASICs), and/or coarse-grained reconfigurable architectures (CGRAs). Deep learning processors 1078 can be hosted by a deep learning cloud platform such as Google Cloud Platform™, Xilinx™, and Cirrascale™. Examples of deep learning processors 1078 include Google's Tensor Processing Unit (TPU)™, rackmount solutions like GX4 Rackmount Series™, GX36 Rackmount Series™ NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, and others.

Memory subsystem 1022 used in the storage subsystem 1010 can include a number of memories including a main random-access memory (RAM) 1032 for storage of instructions and data during program execution and a read-only memory (ROM) 1036 in which fixed instructions are stored. A file storage subsystem 1036 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1036 in the storage subsystem 1010, or in other machines accessible by the processor.

Bus subsystem 1055 provides a mechanism for letting the various components and subsystems of computer system 1000 communicate with each other as intended. Although bus subsystem 1055 is shown schematically as a single bus, alternative implementations of the bus subsystem can use multiple busses.

Computer system 1000 itself can be of varying types including a personal computer, a portable computer, a workstation, a computer terminal, a network computer, a television, a mainframe, a server farm, a widely distributed set of loosely networked computers, or any other data processing system or user device. Due to the ever-changing nature of computers and networks, the description of computer system 1000 depicted in FIG. 15 is intended only as a specific example for purposes of illustrating the preferred implementations of the present invention. Many other configurations of computer system 1000 are possible having more or less components than the computer system depicted in FIG. 15.

We describe various implementations of epigenetic predictors and the training thereof. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

In one implementation, we disclose a method of generating an epigenetic age prediction. The method includes receiving a first sequence of inputs corresponding to methylation values at a plurality of CpG sites of an individual. The method includes receiving a second sequence of inputs corresponding to phenotypic values relative to the individual. The method includes applying a model on the first sequence of inputs and the second sequence of inputs to predict the epigenetic age.

In one implementation, the second sequence of inputs in the method corresponds to phenotypic values of a phenotypic input type.

In one implementation, the phenotypic values of the phenotypic input type include image data obtained from an image of the individual.

In one implementation, the phenotypic values of the phenotypic input type include biometric data of the individual.

In one implementation, the biometric data in the method includes a plurality of values corresponding to one or more of the following input subtypes: oxygen-consumption rate, basal metabolic rate, waist circumference, basal metabolic index, fat percentage, muscle percentage, lean muscle mass, visceral fat mass, and bone mass.

In one implementation, the phenotypic values of the phenotypic input type include cardiovascular data of the individual.

In one implementation, the cardiovascular data includes a plurality of values corresponding to one or more of the following input subtypes: heart rate variability, maximum heart rate, minimum heart rate, heart rate range, and resting heart rate.

In one implementation, the phenotypic values of the phenotypic input type in the method include blood data obtained from a blood sample.

In one implementation, the blood data includes a plurality of values corresponding to one or more of the following input subtypes: blood pressure, blood oxygen levels, lipid profile, sugar profile, and hormone levels.

In one implementation, the input subtype corresponding to a lipid profile includes values of one or more of the following phenotypic measurements: total cholesterol, low-density lipoproteins, high-density lipoproteins, and triglycerides.

In one implementation, the input subtype corresponding to a sugar profile includes values of one or more of the following phenotypic measurements: glucose and hemoglobin A1C.

In one implementation, the input subtype corresponding to hormone levels includes values of one or more of the following phenotypic measurements: cortisol, testosterone, and estrogen.

In one implementation, the second sequence of inputs corresponds to phenotypic values of a plurality of phenotypic input types.

In one implementation, the phenotypic values include values from two or more of the following phenotypic input types: image data, cardiovascular data, biometric data, and blood data.

In another implementation, we disclose a method of generating an epigenetic clock predictor. The method includes receiving a plurality of methylation profiles from a plurality of individuals, the plurality of methylation profiles comprising methylation values for m CpG sites. The method includes receiving a plurality of phenotypic profiles, the plurality of phenotypic profiles comprising phenotypic values for one or more phenotypic input types. The method includes training a model based on the plurality of methylation profiles and the plurality of phenotypic profiles, the model being configured to predict an epigenetic age based on methylation values for n CpG sites and the phenotypic values for the one or more phenotypic input types.

In one implementation, the plurality of phenotypic profiles in the method includes phenotypic values corresponding to one or more of the following input types: image data, cardiovascular data, biometric data, and blood data.

In one implementation, the plurality of phenotypic profiles in the method is received from a plurality of different individuals than the plurality of methylation profiles.

In another implementation, we disclose an epigenetic age predictor. The epigenetic age predictor includes an input component configured to receive a first sequence of inputs corresponding to methylation values at CpG sites of an individual and a second sequence of inputs corresponding to phenotypic values of the individual. The second sequence of inputs in the epigenetic age predictor include phenotypic values corresponding to image data obtained from an image of the individual, the image data being indicative of one or more phenotypic characteristics of the individual. The epigenetic age predictor predicts an epigenetic age of the individual based on the first sequence of inputs and the second sequence of inputs.

In one implementation, the one or more phenotypic characteristics include characteristics relating to the skin of the individual.

In one implementation, the image is a facial image of the individual.

Although the present invention has been described with reference to preferred embodiments, workers skilled in the art will recognize that changes may be made in form and detail without departing from the spirit and scope of the invention.

1. A computer-implemented method of generating an epigenetic age prediction, the method comprising:
receiving a first sequence of inputs corresponding to methylation values at a plurality of CpG sites of an individual;
receiving a second sequence of inputs corresponding to phenotypic values relative to the individual; and
applying a model on the first sequence of inputs and the second sequence of inputs to predict the epigenetic age, wherein the model includes at least one of a trained neural network, fitted linear regression or trained random forest.

2. The method of claim 1, wherein the second sequence of inputs corresponds to phenotypic values of a phenotypic input type.

3. The method of claim 2, wherein the phenotypic values of the phenotypic input type include image data obtained from an image of the individual.

4. The method of claim 2, wherein the phenotypic values of the phenotypic input type include biometric data of the individual.

5. The method of claim 4, wherein the biometric data includes a plurality of values corresponding to one or more of the following input subtypes: oxygen-consumption rate, basal metabolic rate, waist circumference, basal metabolic index, fat percentage, muscle percentage, lean muscle mass, visceral fat mass, and bone mass.

6. The method of claim 2, wherein the phenotypic values of the phenotypic input type include cardiovascular data of the individual.

7. The method of claim 6, wherein the cardiovascular data includes a plurality of values corresponding to one or more of the following input subtypes: heart rate variability, maximum heart rate, minimum heart rate, heart rate range, and resting heart rate.

8. The method of claim 2, wherein the phenotypic values of the phenotypic input type include blood data obtained from a blood sample.

9. The method of claim 8, wherein the blood data includes a plurality of values corresponding to one or more of the following input subtypes: blood pressure, blood oxygen level, lipid profile, sugar profile, and hormone levels.

10. The method of claim 9, wherein the input subtype corresponding to a lipid profile includes values of one or more of the following phenotypic measurements: total cholesterol, low-density lipoproteins, high-density lipoproteins, and triglycerides.

11. The method of claim 9, wherein the input subtype corresponding to a sugar profile includes values of one or more of the following phenotypic measurements: glucose and hemoglobin A1C.

12. The method of claim 9, wherein the input subtype corresponding to hormone levels includes values of one or more of the following phenotypic measurements: cortisol, testosterone, and estrogen.

13. The method of claim 1, wherein the second sequence of inputs corresponds to phenotypic values of a plurality of phenotypic input types.

14. The method of claim 13, wherein the phenotypic values include values from two or more of the following phenotypic input types: image data, cardiovascular data, biometric data, and blood data.

15. A computer-implemented method of generating an epigenetic clock predictor, the method comprising:
receiving a plurality of methylation profiles from a plurality of individuals, the plurality of methylation profiles comprising methylation values for m CpG sites;
receiving a plurality of phenotypic profiles, the plurality of phenotypic profiles comprising phenotypic values for one or more phenotypic input types; and
training a model based on the plurality of methylation profiles and the plurality of phenotypic profiles, the model being configured to predict an epigenetic age based on methylation values for n CpG sites and the phenotypic values for the one or more phenotypic input types, wherein the model includes at least one of a trained neural network, fitted linear regression or trained random forest.

16. The method of claim 15, wherein the plurality of phenotypic profiles includes phenotypic values corresponding to one or more of the following input types: image data, cardiovascular data, biometric data, and blood data.

17. The method of claim 15, wherein the plurality of phenotypic profiles is received from a plurality of different individuals than the plurality of methylation profiles.

18. A computer-implemented epigenetic age predictor comprising:
an input component configured to receive a first sequence of inputs corresponding to methylation values at CpG sites of an individual and a second sequence of inputs corresponding to phenotypic values of the individual;
wherein the second sequence of inputs include phenotypic values corresponding to image data obtained from an image of the individual, the image data being indicative of one or more phenotypic characteristics of the individual; and
wherein the epigenetic age predictor applies at least one of a trained neural network, fitted linear regression or trained random forest to predict an epigenetic age of the individual based on the first sequence of inputs and the second sequence of inputs.

19. The system of claim 18, wherein the one or more phenotypic characteristics include characteristics relating to the skin of the individual.

20. The system of claim 18, wherein the image is a facial image of the individual.

21. The method of claim 1, further including in the first sequence of inputs at least 42 and less than 200 CpG sites of the individual.
